(19) United States
(12) Patent Application Publication
(10) Pub. No.: US 2006/0270019 A1
(43) Pub. Date: Nov. 30, 2006

(54) Method for producing lactic acid bacterium culture containing bacteriocin and a method for preserving food products for by using it

(75) Inventors: Akinori Uehara, Kawasaki-shi (JP); Yasuhiko Toride, Kawasaki-shi (JP)

Correspondence Address: C. IRVIN MCCLELLAND;OBLON, SPIVAK, MCCLELLAND, MAIER & NEUSTADT, P.C., 1940 DUKE STREET, ALEXANDRIA, VA, 22314, US

(73) Assignee: Ajinomoto Co., Inc., Tokyo (JP)

(21) Appl. No.: 11/418,217

(22) Filed: May 5, 2006

Related U.S. Application Data
(63) Continuation of application No. PCT/JP04/16783, filed on Nov. 5, 2004.

(30) Foreign Application Priority Data
Nov. 7, 2003 (JP) 378327/2003

(51) Int. Cl. C12N 1/20; C12P 1/04

(52) U.S. Cl. 435/252.9; 435/170

(57) ABSTRACT

The present invention provides a lactic acid bacterium culture containing protease-resistant bacteriocin, which can be produced by cultivating lactic acid bacteria (e.g., from the genus Weissella). As such, the shelf stability of a food can be improved by incorporating the culture of the present invention therein.

### CROSS-REFERENCE TO RELATED APPLICATIONS

The present application is a continuation of PCT/JP04/016783, filed on Nov. 5, 2004, which claims priority to Japanese Application No. JP 2003-378327, filed on Nov. 7, 2003, which are hereby incorporated by reference in their entirety.

### BACKGROUND OF THE INVENTION

1. Field of the Invention

The present invention provides a method for producing lactic acid bacterium culture containing bacteriosin, a method for preserving food products using the lactic acid bacterium culture containing bacteriocin, and a screening method for a lactic acid bacterium producing bacteriocin.

2. Discussion of the Background

To prevent decay and quality deterioration, food preservatives have been added to various food products. Heretofore, the chemically synthesized food preservatives have been the primary food additive to achieve this goal. However, safety issues (including persistence and toxicity) have been raised with respect to chemically synthesized food preservatives. To solve these problems, there is a desire to develop a safe antimicrobial substance derived from traditional foods.

Lactic acid bacteria are useful microorganisms, which have been traditionally used in the production of various fermented food products (including fermented beverages) such as soy sauce, soybean paste (miso), pickles and Japanese sake. Additionally, lactic acid bacteria have been employed for the production of fermented food products. This is ascribed to the inhibition of the growth of contaminated bacteria in the production process and the resulting products. Due to the pH reduction of the systems with lactic acid produced via lactic acid fermentation, decay and quality deterioration of such food products can be prevented and/or reduced. Additionally, it has been determined that antimicrobial substances produced by lactic acid bacteria are useful for the prevention of the decay and quality deterioration of food products.

Bacteriocin is a proteinaceous antimicrobial substance produced by various bacteria.

Among varieties of bacteriocin produced by lactic acid bacteria, nisin is approved as a GRAS substance by the FDA and has been approved as a safe substance possessing antimicrobial activity by WHO and FAO. Further, nisin is utilized as a food preservative in 50 countries or more all over the world.

Disadvantageously, known varieties of bacteriocin including nisin are readily degraded by proteases. For example, bacteriocin is readily degraded by proteases produced by Aspergillus oryzae (etc.) during the production process of fermented food products like sake, soy sauce and soybean paste (miso). Therefore, the bacteriocin cannot maintain satisfactory antimicrobial activities. In the case of nisin addition prior to pasteurization for sake (Publication No.JP06-319516) and in the case of the addition of acidocin 8912 as another bacteriocin variety (Publication No.JP06-319516), it is reported that antimicrobial effects cannot be obtained because these varieties of bacteriocin are degraded by proteolytic enzymes present in the unprocessed sake. Currently, no report exists indicating that lactic acid bacteria produce protease-resistant bacteriocin.

Processed meat products, such as ham and sausage, are spontaneously fermented due to microorganisms inherently existing in the raw materials thereof or microorganisms contaminated during the production process, so that preferable flavor and preservability can be imparted thereto. Currently, the starter culture method has been developed (Science and Technology of Lactic Acid Bacteria, Association Press Center, p. 239, 1996) to stabilize product quality, shorten production times, and prevent growth of hazardous microorganisms.

For example, Chung et al. disclose the effect of immersing uncooked meat in nisin solution or the effect of nisin on fresh edible meat preliminarily inoculated with certain bacterial species. According to the report, nisin used in fresh edible meat loses its activity in a very short time (Env. Microbiol. 55: (6) p. 1329-1333 (1989)). This is due to the nisin decomposition with protease such as cathepsin in edible meat causing the loss of the antimicrobial activity in a very short time.

Therefore, to prevent nisin decomposition with enzymes as described above, an invention is reported, which includes heat-treating edible meat and subsequently applying lanthionine-base bacteriocin such as nisin to the surface of the heat-treated edible meat (Publication No.JP06-22685). However, edible meat should be heat-treated before nisin addition. Therefore, the range of the use of the invention is more or less limited.

Therefore, in the modern food industries, there exists a critical need for the development of a protease-resistant bacteriocin applicable to a wide range of food products including fermented food products and processed meat products.

Armenia is known as a country where people enjoy longer longevity. In Armenia, traditionally, a great number of healthy food products have been formulated for sickness. For example, lactic acid-containing food products, such as Matsoon and Narine, dry apricot, red wine, jyesiin and tiinaff. have been prepared. Therefore, based on fermented milk Matsoon eaten in Armenia and koji as a raw material for fermented food products, the present inventors have sought to address the foregoing critical need.

### SUMMARY OF THE INVENTION

As described above, lactic acid bacteria have been used traditionally for the treatment of various food products including fermented food products and have never caused any safety concerns. Additionally, antimicrobial substances produced by lactic acid bacteria are considered to be safer than chemically synthesized substances. Thus, the objectives of the invention are to provide 1) a method for producing lactic acid bacterium culture containing protease-resistant bacteriocin; 2) a method for preserving food products using the culture containing the said bacteriocin; and 3) a screening method for a lactic acid bacterium producing protease-resistant bacteriocin.

In order to solve the problems, the inventors isolated lactic acid bacteria existing in fermented food products such as fermented milk, and screen bacterial strains which produce novel protease-resistant bacteriocin. Consequently, the inventors successfully isolated a lactic acid bacterium producing the substance. The inventors confirmed that the bacteriocin produced by the bacterial strain is a novel substance. And, the invention is described as follows.

An object of the present invention is to provide a method for producing a lactic acid bacterium culture containing bacteriocin which is resistant to proteases by culturing the lactic acid bacterium for a time and under conditions suitable for expressing said bacteriocin which is resistant to proteases in a suitable medium for said culturing.

In an embodiment of this object, the lactic acid bacterium may belong to a genus selected from the group consisting of Weissella, Pediococcus, Lactobacillus and Leuconostoc.

In another embodiment of this object, the method may further entail recovering the lactic acid bacterium and isolating the bacteriocin which is resistant to proteases.

In another object of the present invention is to provide a method for preserving a food product, by mixing the lactic acid bacterium culture or bacteriocin which is resistant to proteases (infra) with a food product during the production thereof.

In various embodiments of the foregoing object, the lactic acid bacterium culture or bacteriocin which is resistant to proteases may be either mixed with the starting materials for production of the food product or may be added to the final prepared food product. Envisioned food products include fermented food products or processed meat products.

It is yet another object of the present invention to provide a method for screening a lactic acid bacterium which produces bacteriocin which is resistant to proteases by

(a) culturing the lactic acid bacterium for a time and under conditions suitable for expressing a bacteriocin in a suitable medium for said culturing to obtain a lactic acid bacterium culture;

(b) assessing the antimicrobial activity of the lactic acid bacterium culture in the presence of a protease (e.g., a protease derived from Aspergillus oryzae); and

(c) classifying the lactic acid bacterium obtained by (a) as resistant to proteases where the culture forms an inhibitory zone in (b).

It is still another object of the present invention to provide a novel bacterial strain defined as Weissella sp. AJ110263 (FERM BP-10474).

The above objects highlight certain aspects of the invention. Additional objects, aspects and embodiments of the invention are found in the following detailed description of the invention.

### DETAILED DESCRIPTION OF THE INVENTION

Unless specifically defined, all technical and scientific terms used herein have the same meaning as commonly understood by a skilled artisan in enzymology, biochemistry, cellular biology, molecular biology, and the medical sciences.

All methods and materials similar or equivalent to those described herein can be used in the practice or testing of the present invention, with suitable methods and materials being described herein. All publications, patent applications, patents, and other references mentioned herein are incorporated by reference in their entirety. In case of conflict, the present specification, including definitions, will control. Further, the materials, methods, and examples are illustrative only and are not intended to be limiting, unless otherwise specified.

In the present invention, the phrases “bacteriocin which is resistant to proteases” or “protease-resistant bacteriocin” refers to a bacteriocin which has an antimicrobial activity even in the presence of a protease. An exemplary protease is that derived from Aspergillus oryzae. Bacteriocin whose antimicrobial activity is reduced by amylases is also included.

The lactic acid bacterium culture containing “bacteriocin which is resistant to proteases” or “protease-resistant bacteriocin” refers to a culture which forms an inhibitory zone of the indicator strain in the following method more specifically:

(1) A lactic acid bacterium culture is prepared according to an ordinary cultivation method (or a cultivation method for separating microorganisms). The pH of the lactic acid bacterium culture is adjusted to pH5.5 to 6.0 with sodium hydroxide solution. Subsequently, the culture is centrifuged at 12,000 rpm for 10 minutes and filtrated with Disposable Syringe Filter Unit “Dismic-25cs”, Cellulose Acetate 0.45 μm (ADVANTEC Inc). The filtered liquid is used as a sample. If the antimicrobial activity of the sample is low, the sample needs to be concentrated up to (and including) 4 times under reduced pressure at ambient temperature. If necessary, it is concentrated up to (and including) 10 times.

(2) Listeria innocua ATCC33090T, Bacillus circulans JCM2504T, Bacillus coagulans JCM2257, Micrococcus luteus IF012708, Bacillus subtilis JCM1465T, Bacillus subtilis IAM1381, Lactococcus lactis sub sp. Lactis ATCC19435, Enterococcus faecium JCM5804T, Enterococcus faecium JCM5803T, Lactobacillus plantarum ATCC14917T and Lactobacillus sakei JCM1157T are used as an inidicator strain. The indicator having the highest antimicrobial activity is selected by measuring the antimicrobial activities by the spot-on-lawn method (supra) or counting the colony forming unit.

(3) A protease derived from Aspergillus (Umamizyme G, Amano Enzyme Co) is used as an enzyme.

(4) 10 to 100 unit/ml of the protease described in (3) is added to the sample described in (1) and reacted at 30° C. for more than one hour.

(5) The indicator strain exhibiting the highest antimicrobial activity described in (2) is spread on a medium plate (e.g., an MRS medium plate) where the indicator can grow. 0.01 ml of the protease treated sample described in (4) is dropped on the center of the medium plate at the optimal temperature for the growth of the indicator (e.g., 37° C. for Listeria innocua, Bacillus coagulans, Enterococcus faecium or Pediococcus pentosaceus and 30° C. for others) for 20 to 24 hours. Then, the inhibitory zone of the indicator is observed.

Lactic acid bacteria that produce bacteriocin resistant to proteases according to the present invention are separated from fermented food products and so on. It is needless to say that lactic acid bacteria with antimicrobial activity obtainable by the screening method described below may be used as well.

In other words, any lactic acid bacteria producing protease-resistant bacteriocin may be used within the context of the present invention, with no specific limitation to the source from which the bacteria are separated. As a result of examinations by the inventors, the inventors discovered that among lactic acid bacteria, genera Weissella, Pediococcus, Lactobacillus, Leuconostoc, and so on produce the intended protease-resistant bacteriocin. Strains warranting specific mention are the Weissella strains: Weissella sp. FERM BP-10474,Weissella cibaria JCM12495, Wissella confusa JCM1093, Weissella hellenica JCM10103, Weissella kandleri JCM5817, Weissella minor JCM1168, Weissella paramesenteroides JCM9890, and Weissella thailandensis JCM10694; the Pediococcus strain Pediococcus pentosaceus; the Lactobacillus strains Lactobacillus plantarum, Lactobacillus salivarius, and Lactobacillus pentosus; and the Leuconostoc strains Leuconostoc citreum, Leuconostoc pseudomesenteroides, Leuconostoc argentinum, Leuconostoccamosum, and Leuconostoc mesenteroides. However, the present invention also embraces lactic acid bacteria other than those expressly described herein, so long as the lactic acid bacteria produce protease-resistant bacteriocin.

In accordance with the present invention decay and quality deterioration of the intended food products can be prevented and/or reduced by using the lactic acid bacterium culture containing protease-resistant bacteriocin obtained by cultivating lactic acid bacteria producing the protease-resistant bacteriocin. In this process, exemplary food products are fermented food products such as soy sauce, miso and fish sauce and various types of processed meat products such as ham and sausage.

The bacteriocin may be isolated and used. Otherwise, the culture containing bacteriocin may be used as it is, with no isolation of the bacteriocin. Because purification procedures such as isolation are generally laborious, preferably, the lactic acid bacterium culture itself is added in a process of producing various types of fermented food products. Further, the culture containing the protease-resistant bacteriocin may satisfactorily be added in one portion or plural portions, in a process of producing fermented food products, processed meat products and so on. Satisfactorily, how many portions the bacteriocin or the broth is divided into for addition may be freely determined.

To obtain the intended lactic acid bacterium culture containing protease-resistant bacteriocin, the lactic acid bacteria should be cultivated. Cultivation conditions such as cultivation temperature, culture time, cultivation method and medium are not particularly limiting and may be the ordinary condition used in cultivating lactic acid bacteria. Additionally, routine separation and purification methods such as gel filtration may be used for isolation.

The lactic acid bacterium culture or the lactic acid bacteria culture in the present invention refers to a medium containing cultivated lactic acid bacteria or a medium which cultivated lactic acid bacteria is removed from by centrifuge or the like. And, the medium may be liquid, solid or gel-like. When the medium is a liquid medium, it is sometimes described as a lactic acid bacterium broth. The lactic acid bacterium culture also embraces a lactic acid bacterium broth. In addition, a dried powder of liquid lactic acid bacterium/bacteria culture by spray drying, freeze-drying or the like, a concentrated liquid or paste of liquid lactic acid bacterium/bacteria culture by filtration, evaporation or the like, or a fraction with antimicrobial activities of liquid lactic acid bacterium/bacteria culture by gel filtration, chromatography or the like is also included as lactic acid bacterium/bacteria culture of this invention.

The lactic acid bacterium culture containing protease-resistant bacteriocin may be added to any food products, with no specific limitation. Most preferably, the culture is added to fermented food products and processed meat products where microorganisms are involved in their production process.

Fermented food products include soy sauce, fish sauce, sake, soybean paste miso, pickles, cheese and so on. These are just examples. The lactic acid bacterium culture containing protease-resistant bacteriocin may satisfactorily be used for those other than the examples described above.

Herein, traditionally, sodium chloride has been used as a bacteriostatic agent in fermented food products. Owing to the increase of demands toward low salt diet in recent years and the advantage of expediting the protein decomposition rate by lowering or removing salt in the fermentation, research has been made to use bacteriocin such as nisin in fermented food products. Because nisin and the existing varieties of bacteriocin are decomposed by proteases existing in the production processes, however, the bacteriostatic effect is not currently observed. Even for the processes of producing fermented food products, the lactic acid bacterium culture containing bacteriocin with protease resistance can be used.

Additionally, the processed meat products include, for example, ham and sausage. Therefore, the culture containing the protease-resistant bacteriocin may satisfactorily be used for those other than the examples just described above.

Food products such as fermented food products and processed meat products produced by the addition of the lactic acid bacterium culture containing protease-resistant bacteriocin have extremely high shelf stability.

The screening method for a lactic acid bacterium producing the protease-resistant bacteriocin as an important aspect of the invention is now described in the following example where such lactic acid bacterium is separated from a fermented food product Matsoon.

A sample collected from fermented milk Matsoon which is one of fermented food products is cultivated in a medium where a lactic acid bacterium can grow, for example the MRS medium (Table 1) or the M17 medium (Table 2) at 30° C. to 37° C., whrein the amount of the sample to the medium is 0.5%. The culture time is one day, 5 days and 10 days. After completion of the cultivation, the broth is spread and cultivated on the agar medium (agar at 1.2%) containing 0.5% calcium carbonate. From the resulting colonies, lactic acid bacteria are collected.

TABLE 1
Composition of MRS medium
Composition of MRS medium (Merck)

| | |
|---|---|
| Peptone | 10.0 g/l |
| Lab-Lemco's Powder | 8.0 g/l |
| Yeast extract | 4.0 g/l |
| Glucose | 20.0 g/l |
| Tween 80 | 1.0 g/l |
| Dipotassium hydrogen phosphate | 2.0 g/l |
| Sodium acetate | 5.0 g/l |
| Ammonium citrate | 2.0 g/l |
| Magnesium sulfate · 7H₂O | 0.20 g/l |

TABLE 2
Composition of M17 medium
Composition of M17 medium (Merck)

| | |
|---|---|
| Soybean meal-derived peptone | 5.0 g/l |
| Meat-derived peptone | 2.5 g/l |
| Casein-derived peptone | 2.5 g/l |
| Yeast extract | 2.5 g/l |
| Meat extract | 5.0 g/l |
| D(+)-Lactose | 5.0 g/l |
| Ascorbic acid | 0.5 g/l |
| β-Glycerophosphate sodium | 19.0 g/l |
| Magnesium sulfate | 0.25 g/l |

The collected lactic acid bacteria are cultivated in the heretofore described manner. Then, the lactic acid bacteria are inoculated and cultivated for 24 hours on a plate of MRS agar medium to which filtrated Umamizyme G (a protease derived from Aspergillus oryzaeproteases; Amano Enzyme Co) is added. Subsequently, the Lactobacilli AOAC medium (Table 3) into which an indicator strain is initially mixed is overlaid on the plate and cultivated for 24 hours, to form an inhibitory zone of the indicator strain.

TABLE 3
Composition of Lactobacilli AOAC medium
Composition of Lactobacilli AOAC medium (Difco)

| | |
|---|---|
| Peptonized milk | 15.0 g/l |
| Yeast extract | 5.0 g/l |
| Dextrose | 10.0 g/l |
| Tomato juice | 5.00 g/l |
| Monopotassium dihydrogen phosphate | 2.0 g/l |
| Polysorbate 80 | 1.0 g/l |

For adding the protease, several methods may be employed in addition to a method of mixing the protease into the agar medium. These methods include:

1) mixing the protease with an indicator strain into the medium;

2) spreading the protease on the agar medium;

3) adding the protease when the colonies of lactic acid bacteria are cultivated. In this case, the protease may be added at the start of cultivation, during cultivation or on the completion of cultivation; and,

4) observing the formation of the inhibitory zone by adding onto a plate, where an indicator strain is mixed, an appropriate amount of the sample where the protease was added after cultivating colonies of lactic acid bacteria and then disinfecting or killing the bacteria in the broth.

However, the present invention is not limited to the methods 1) to 4). Additionally, the protease is not limited to Umamizyme G.

Then, antimicrobial spectral analysis is performed. Using the spot-on-lawn method, the supernatant of the lactic acid bacterium culture with antimicrobial activities is spotted on a plate and is examined as described below.

First, a sample with antimicrobial activity is prepared. The culture liquid of the bacterial strain having an antimicrobial activity obtained by the aforementioned method is centrifuged at 10,000 rpm for 10 minutes to obtain a culture supernatant. And then the supernatant is filtrated through a filter to obtain an aseptic sample. The sample is diluted by every 2 fold to prepare a dilution series to 2¹¹ dilutions. In case that the activity is low, the sample is concentrated by every 2 fold to prepare a concentration series to 2⁻³ dilutions under reduced pressure at ambient temperature.

Then, the indicator strain to be mixed on the plate for examining the antimicrobial activity is cultivated. The indicators in Table 4 are cultivated in the TSBYE medium (Tables 5 and 6) or the MRS medium. Bacteria of the genera Bacillus and Micrococcus are cultivated by using a shaker but the other bacteria are statically cultivated. Additionally, Bacillus coagulans, Listeria, Pediococcus and Enterococcus are cultivated at 37° C., while the other are cultivated at 30° C.

TABLE 4
Indicator strain for evaluating Antimicrobial activity

| | Medium/ | |
|---|---|---|
| | Temperature | Cultivation |
| Name of bacterial strain | (° C.) | method |
| Bacillus coagulans | TSBYE/37 | Shaker culture |
| JCM2257 | | |
| Bacillus subtilis | TSBYE/30 | Shaker culture |
| JCM1465T | | |
| Bacillus subtilis | TSBYE/30 | Shaker culture |
| IAM1381 | | |
| Bacillus circulans | TSBYE/30 | Shaker culture |
| JCM2504T | | |
| Micrococcus luteus | TSBYE/30 | Shaker culture |
| IFO12708 | | |
| Listeria innocua | TSBYE/37 | Static culture |
| ATCC33090T | | |
| Pediococcus pentosaceus | MRS/37 | Static culture |
| JCM5885 | | |
| Enterococcus faecalis | MRS/37 | Static culture |
| JCM5803T | | |
| Enterococcus faecium | MRS/37 | Static culture |
| JCM5804T | | |
| Lactococcus lactis subsp. | MRS/30 | Static culture |
| lactis ATCC19435 | | |
| Lactobacillus plantarum | MRS/30 | Static culture |
| ATCC14917T | | |
| Lactobacillus sakei subsp. | MRS/30 | Static culture |
| sakei JCM1157T | | |
| Leuconostoc mesenteroides | MRS/30 | Static culture |
| subsp. mesenteroides | | |
| JCM6124T | | |
| Lactobacillus kimchii | MRS/30 | Static culture |
| JCM10707T | | |

TABLE 5
Composition of TSBYE medium
Composition of TSBYE medium

| | |
|---|---|
| TSB medium | 30.0 g/l |
| Yeast extract (, Difco) | 6.0 g/l |

TABLE 6
Composition of TSB medium
Composition of Bacto tryptic soy broth (TSB) medium (Difco)

| | |
|---|---|
| Pancreatic digest of casein | 17.0 g/l |
| Enzymatic digest of soybean meal | 3.0 g/l |
| Dextrose | 2.5 g/l |
| Sodium chloride | 5.0 g/l |
| Dipotassium monohydrogen phosphate | 2.5 g/l |

Further, a plate for examining the antimicrobial activity is prepared. 10 ml of the MRS agar medium (agar at 1.2%) and 5 ml of the Lactobacilli AOAC agar medium (agar at 1.2%) are separately sterilized at 121° C. for 15 minutes and are then kept warm at 55° C. The sterilized MRS agar medium is poured into an aseptic petri dish and is incubated on a clean bench for one hour. Subsequently, 50 μl of a broth of the indicator strain is mixed to the Lactobacilli AOAC agar medium kept warm at 55° C. The broth is overlaid on the MRS plate. The lid of the plate is opened in the clean bench (for about 15 minutes), to dry the surface.

10-μl each of the prepared sample with the antimicrobial activity as prepared above is dropped to the plate. Then, the lid is closed and the plate is incubated for about one hour, to dry the plate. The plate is incubated at a suitable temperature of each indicator strain for 20 hours, to examine the formation of an inhibitory zone. Herein, the antimicrobial activity (AU/ml) is defined as follows.
Antimicrobial activity (AU/ml)=(maximum dilution ratio for forming the inhibitory circle)×1000/10

The samples of which the antimicrobial spectrum was analyzed in such manner had protease resistance and showed a wide range of antimicrobial spectrum.

The bacteriological profile of the lactic acid bacterial strain AJ110263 selected by the method described above was examined. Based on the homology analysis in terms of the nucleotide sequence of 16S ribosome DNA (rDNA) (Altschul, S. F., Madden T. F., Schaffer, A. A., Zhang, J., Zhang, Z., Miller, W., and Lipman, D. J. (1997) Gapped BLAST and PSI-BLAST: a new generation of protein database search programs. Nucleic Acids Res. 25: 3389-3402.), the bacterial strain had 98.22% homology to Weissella confusa strain ATCC 10881 (Table 7). For the homology assessment, herein, the type culture which is deposited at ATCC was used.

TABLE 7
16S rDNA homology of strain AJ110263

| | | |
|---|---|---|
| 16S rDNA homology | Weissella confusa | 98.22% |
| | Weissella viridescens | 95.20% |
| | Weissella minor | 92.54% |
| | Weissella kandleri | 92.01% |
| | Weissella halotolerans | 87.39% |
| | Weissella | 86.25% |
| | paramesenteroide | |
| | Lactobacillus mali | 78.17% |
| | Pediococcus parvulus | 77.58% |

It was considered that the basic profile (Table 8) of the bacterial strain coincided with the general properties of lactic acid bacteria and that the sugar fermentation pattern (Table 9) was similar to that of Weissella confusa. However, the bacterial strain showed a different fermentation pattern for L-arabinose and did not have 100% homology on the basis of 16S rDNA. Therefore, it was determined that this bacterial strain is a novel bacterial strain different from any known bacteria. Thus, the bacterial strain was defined as Weissella sp. AJ110263 and was deposited at the International Patent Organism Depositary (IPOD), the National Institute of Advanced Industrial Science and Technology (AIST). Its accession number is FERM BP-10474.

TABLE 8
Basic profile of Lactic acid bacterium strain AJ110263

| | Short bacillus |
|---|---|
| Cell morphology | (0.8-1.0 × 1.0-1.5 μm) |
| Gram staining | (+) |
| Spore | (−) |
| Mobility | (−) |
| Colony morphology | circle |
| (medium: MRS medium) | overall periphery smooth |
| (cultivation temperature: 30° C.) | lowly protruded shape |
| (culture time: 24 hr) | gloss opaque |
| Cultivation temperature | (+/−) |
| (37° C./45° C.) | |
| Catalase | (−) |
| Acid/gas generation (glucose) | (+/−) |
| O/F test (glucose) | (+/+) |
| Growth at pH 9.6 | (+) |
| Growth in NaOH (6.5%) | (+) |

TABLE 9
Fermentation profile of sugars of
Lactic acid bacterium strain AJ110263
Fermentation profile of sugars

| | | |
|---|---|---|
| Fermentation (+) | L-arabinose | arbutin |
| | D-xylose | esculin |
| | Galactose | salicin |
| | Glucose | cellobiose |
| | Fructose | maltose |
| | Mannose | purified sugar |
| | N-acetylglucosamine | gentiobiose |
| | Amygdalin | gluconate |
| Fermentation (−) | Glycerol | trehalose |
| | Erythritol | inulin |
| | D-arabinose | melezitose |
| | Ribose | raffinose |
| | L-xylose | starch |
| | Adonitol | glycogen |
| | β-methyl-D-xylose | xylitol |
| | Sorbose | D-tulanose |
| | Rhamnose | D-lyxose |
| | Dulcitol | D-tagatose |
| | Inositol | D-fucose |
| | Mannitol | L-fucose |
| | Sorbitol | D-arabitol |
| | α-methyl-D-mannose | L-arabitol |
| | α-methyl-D-glucose | 2-ketogluconic acid |
| | Lactose | 5-ketogluconic acid |
| | Melibiose | |

The present invention provides a method for preserving food products by adding a lactic acid bacterium culture containing protease-resistant bacteriocin produced by lactic acid bacteria. In an embodiment of the present invention, the lactic acid bacteria is selected from Weissella sp., Pediococcus pentosaceus, Lactobacillus plantarum and Lactobacillus salivarius. Further, the present invention provides a manufacturing process for fermented food products, processed meat products, etc.

The above written description of the invention provides a manner and process of making and using it such that any person skilled in this art is enabled to make and use the same, this enablement being provided in particular for the subject matter of the appended claims, which make up a part of the original description.

As used above, the phrases “selected from the group consisting of,” “chosen from,” and the like include mixtures of the specified materials.

Where a numerical limit or range is stated herein, the endpoints are included. Also, all values and subranges within a numerical limit or range are specifically included as if explicitly written out.

The above description is presented to enable a person skilled in the art to make and use the invention, and is provided in the context of a particular application and its requirements. Various modifications to the preferred embodiments will be readily apparent to those skilled in the art, and the generic principles defined herein may be applied to other embodiments and applications without departing from the spirit and scope of the invention. Thus, this invention is not intended to be limited to the embodiments shown, but is to be accorded the widest scope consistent with the principles and features disclosed herein.

Having generally described this invention, a further understanding can be obtained by reference to certain specific examples, which are provided herein for purposes of illustration only, and are not intended to be limiting unless otherwise specified.

### EXAMPLES

Weissella sp. AJ110263 (FERM BP-10474) separated from fermented milk Matsoon and Pediococcus pentosaceus JCM5885, Pediococcus pentosaceus JCM5890, Lactobacillus plantarum JCM1149 and Lactobacillus salivarius obtained from the type cultures were initially cultivated followed by cultivation in the MRS medium (Table 1). The Weissella sp. was cultivated at 30° C., while the other bacterial strains were cultivated at 37° C. The lactic acid bacteria were inoculated on the plate of the MRS medium where 0 U/ml (not added), 200 U/ml and 400 U/ml of Umamizyme G shown in Table 3 were added, and cultivated for 24-hour.

Herein, cultivation was carried out by charging 100 ml of the MRS medium in a 500-ml Sakaguchi's flask and subsequently inoculating 100 μl of each of the preliminary broth for cultivation at a shaker of 100 strokes/min.

There after the Lactobacilli AOAC medium where Lactobacillus sakei strain JCM1157 was mixed as an indicator strain, was overlaid. These plates were incubated for 24 hours. Consequently, inhibitory zones of the indicator were formed (Table 10). These results indicated that each strain produces the protease-resistant bacteriocin.

TABLE 10
Diameter(mm) of Inhibitory Zone of PRB producing strain

| | Protease (U/ml) | | |
|---|---|---|---|
| Strain | 0 | 200 | 400 |
| Weissella sp. AJ110263 | 7 | 10 | 10 |
| Pediococcus pentosaceus JCM5885 | 15 | 15 | 15 |
| Pediococcus pentosaceus JCM5890 | 10 | 12 | 12 |
| Lactobacillus plantarum JCM1149 | 15 | 17 | 23 |
| Lactobacillus salivarius JCM1231 | 13 | 18 | 18 |

Lactobacillus sakei strain JCM1157 was used as the indicator strain.
Numeric values in the table express the diameter(mm) of the inhibitory zone.

### Example 2

Lactococcus lactis NCDO497 (a nisinA producer) and Lactococcus lactis NCIMB702054 (a nisin Z producer) were cultivated in the MRS medium at 30° C. In the same manner as in Example 1, the antimicrobial activity was evaluated, using Lactobacillus sakei strain JCM1157 as an indicator strain.

Additionally, the antimicrobial activity was evaluated, by spotting 10 μl of a 1000 IU/ml solution of Nisin A, ICN Biomedical Inc. instead of using the nisin producer, on the plate of the MRS agar medium.

In the absence of protease, an inhibitory zone of the indicator strain was formed. In the presence of protease, the activity was lowered in case the protease concentration was higher (Table 11).

TABLE 11
Diameter(mm) of Inhibitory Zone of Strain not producing PRB

| | Protease (U/ml) | | |
|---|---|---|---|
| Strain | 0 | 200 | 400 |
| Nisin A added | 30 | ND | ND |
| (no use of any bacterial strain) | | | |
| Lactococcus lactis NCDO497 | 30 | 13 | ND |
| (a nisin A producer) | | | |
| Lactococcus lactis NCIMB702054 | 30 | 13 | ND |
| (a nisin Z producer) | | | |

Lactobacillus sakei strain JCM1157 was used as the indicator strain.
Numeric values in the table express the diameter(mm) of the inhibitory zone.
ND = not detected

### Example 3

The strains Weissella sp. AJ110263 (FERM BP-10474), Pediococcus pentosaceus JCM5885, Lactococcus lactis NCDO497 (a nisinA producer) and Lactobacillus sakei JCM1157 were cultivated. The broth was centrifuged at 10,000 rpm for 10 minutes, to obtain culture supernatants. After adding 2000 U/ml of Umamizyme to the supernatants and treating by the enzyme for 24-hour, the supernatants were filtrated with a filter (DISMIC25CS , ADVANTEC; 0.45 μm), to prepare aseptic samples. Using the spot-on-lawn method, the antimicrobial spectra were examined.

Consequently, Weissella sp. AJ110263 (FERM BP-10474) and Pediococcus pentosaceus JCM5885 kept their antimicrobial activities even after the protease treatment, compared with the broth of the nisin-producing bacterium and Lactobacillus sakei JCM1157 which does not produce bacteriocin (Table 12). This indicated that Weissella sp. AJ110263 (FERM BP-10474) and Pediococcus pentosaceus JCM5885 produce the protease-resistant bacteriocin.

TABLE 12

| | Sample | | | |
|---|---|---|---|---|
| | Weissella sp. | Pediococcus | Lactococcus lactis | Lactobacillus sakei |
| Indicator | AJ110263 | pentosaceus JCM5885 | NCDO497 | JCM1157T |
| Listeria innocua | 50 | 50 | ND | ND |
| ATCC33090T | | | | |
| Bacillus circulans | 100 | 100 | 50 | 50 |
| JCM2504T | | | | |
| Bacillus coagulans | 50 | 100 | ND | ND |
| JCM2257 | | | | |
| Micrococcus luteus | 100 | 100 | ND | ND |
| IFO12708 | | | | |
| Bacillus subtilis | 100 | 100 | ND | 50 |
| JCM1465T | | | | |
| Lactococcus lactis | 50 | 50 | ND | ND |
| subsp. lactis ATCC19435 | | | | |
| Enterococcus faecium | 50 | 100 | ND | ND |
| JCM5804T | | | | |
| Enterococcus faecalis | 100 | 100 | ND | 50 |
| JCM5803T | | | | |
| Lactobacillus plantarum | 100 | 100 | ND | 50 |
| ATCC14917T | | | | |
| Lactobacillus sakei | 50 | 50 | ND | ND |
| JCM1157T | | | | |

After the broth was treated with protease, the supernatants were evaluated by the spot-on-lawn method.
Numeric values express antimicrobial activity.
Antimicrobial activity (AU/ml) = maximum dilution ratio for forming inhibitory circle × 1000/10;
ND = not detected.

### Example 4

Culture supernatants of strains Weissella sp. AJ110263 (FERM BP-10474), Pediococcus pentosaceus JCM5885, Lactobacillus plantarum JCM1149, Lactobacillus salivarius JCM1231, Leuconostoc citreum JCM9698, Leuconostoc pseudomesenteroides JCM9696, JCM11045 and Lactococcus lactis NCIMB702054 (a bacterium producing nisin Z) were treated with the enzyme as described in Example 3. Bacillus subtilis IAM1381 was used as an indicator strain. As the enzyme, Umamizyme G derived from Aspergillus oryzae was used as described in Example 3.in addition, α-amylase derived from Bacillus subtilis (Wako Pure Chemical Ltd) was added to the lactic acid bacterium culture broth in an amount of 100 U/ml and submitted to the reaction at 30° C. for more than one hour. Subsequently, the antimicrobial activity was evaluated by the spot-lawn method wherein Bacillus subtilis IAM1381 was used as an indicator strain in the same manner, to study the effect of α-amylase to the antimicrobial activity.

As shown in Table 13, Weisella sp.AJ110263 (FERM BP-10474), Weissella cibaria JCM12495, Weissella confusa JCM1093, Weissella hellenica JCM10103, Weissella kandleri JCM5817, Weissella minor JCM1168, Weissella paramesenteroides JCM9890, Weissella thailandensis JCM10694, Pediococcus pentosaceus JCM5885,Lactobacillus plantarum JCM1149, Lactobacillus salivarius JCM1231, Lactobacillus pentosus JCM1558, Leuconostoc citreum JCM9698, Leuconostoc pseudomesenteroides JCM9696, JCM11045, Leuconostoc argentinum JCM11052, Leuconostoc camosum JCM9695 and Leuconostoc mesenteroides JCM6124 kept their antimicrobial activities even after the protease treatment. Consequently, it was found these strains produced protease resistant bacteriocin.

TABLE 13
Residual Antimicrobial Activity after Enzyme Treatment

| | Residual antibiotic activity | | |
|---|---|---|---|
| | | umami- | α- |
| strain | control | zyme | amylase |
| Nisin producer | | | |
| Lactococcus lactis NCIMB702054 | 100 | nd | 100 |
| Protease-resisitant- | | | |
| bacteriocin (PRB) producer | | | |
| Weissella sp. AJ110263 | 100 | 100 | 30 |
| Weissella cibaria JCM12495 | 100 | 100 | 30 |
| Weissella confusa JCM1093 | 100 | 70 | 50 |
| Weissella hellenica JCM10103 | 100 | 100 | 40 |
| Weissella kandleri JCM5817 | 100 | 100 | 40 |
| Weissella minor JCM1168 | 100 | 100 | 40 |
| Weissella paramesenteroides | 100 | 100 | 70 |
| JCM9890 | | | |
| Weissella thailandensis JCM10694 | 100 | 100 | 40 |
| Pediococcus pentosaceus JCM5885 | 100 | 90 | 30 |
| Lactobacillus plantarum JCM1149 | 100 | 80 | 30 |
| Lactobacillus salivarius JCM1231 | 100 | 80 | 30 |
| Lactobacillus pentosus IAM1558 | 100 | 100 | 30 |
| Leuconostoc citreum JCM9698 | 100 | 80 | 40 |
| Leuconostoc pseudomesenteroides | 100 | 100 | 50 |
| JCM9696 | | | |
| Leuconostoc pseudomesenteroides | 100 | 100 | 50 |
| JCM11045 | | | |
| Leuconostoc argentinum JCM11052 | 100 | 100 | nd |
| Leuconostoc carnosum JCM9695 | 100 | 100 | 30 |
| Leuconostoc mesenteroides JCM6124 | 100 | 100 | 40 |

### Example 5

Soybean (10 g) and pure water (10 ml) were individually added into six Erlenmeyer flasks (200-ml volume) and sterilized in an autoclave at 120° C. for 30 minutes. After cooling, 0.04 g of koji mold(Purple 1, NO.1 bacterium for soy sauce) was added, and cultivated statically at 30° C. for 2 days. 40 ml of sterile pure water were added to cultivated sample (Sample No.1), 40 ml of salt solution which adjusted the salt content of the sample to 18% to Sample No.2, 40 ml of a culture supernatant of Lactococcus lactis NCIMB702054 (the bacterium producing nisin Z) to Sample No.3 and 40 ml of a culture supernatant of Weissella sp. AJ110263 (FERM BP-10474) to Sample No.4, 40 ml of a culture supernatant of Pediococcus pentosaseceus JCM5885 to Sample 5 and 40 ml of a culture supernatant of Lactobacillus salivarius JCM1231 to Sample 6. The resulting mixtures were then adjusted to pH 6.5 to 7.0, using 6N hydrochloric acid and 6N NaOH passed through a filter.

200 μl of Bacillus subtilis IAM1381 cultivated in the TSBYE medium by a shaker at 30° C. for 20 hours was additionally inoculated, mixed thoroughly and cultivated at 30° C. On days 1, 2 and 7 of the cultivation, the broth was collected to count the viable cells of Bacillus subtilis IAM1381 on the GAM agar medium (GAM bouillon “NISSUI”, Nissui Pharmaceutical Co., Ltd.). In the pure water sample, the contaminating bacterium Bacillus subtilis IAM1381 existed at 10⁸ cells per gram or more. In the 18% salt sample, no contamination occurred. In case of the addition of the nisin supernatant with no salt, meanwhile, nisin was decomposed with proteases derived from the koji mold on day 1 and thereafter. Thus, 10⁸ cells per gram or more contaminated therein and no antimicrobial effect was observed.

When using the supernatant of Weissella sp. AJ110263 (FERM BP-10474), Pediococcus pentosaseceus JCM5885 and Lactobacillus salivarius JCM1231, however, the contaminating bacterium Bacillus subtilis LAM1381 was never observed on the first day of the fermentation up to day 7 (Table 14).

TABLE 14
Test to use bacteriosin as a substitute for salt in the manufacturing process of soy sauce

| | | | | On day 1 of | | | |
|---|---|---|---|---|---|---|---|
| | | | | fermentation | | On day 7 | |
| | | Liquid of lactic | | Glu | viable cell | Glu | viable cell |
| No | Salt | acid bacterium | Bacteriosin | (mg/dl) | count BS | (mg/dl) | count BS |
| 1 | not added | absence | not added | 400 | 3 × 10\hat{ }8 | 980 | 3 × 10\hat{ }7 |
| 2 | 18% | absence | not added | 90 | ND | 490 | ND |
| 3 | not added | Lactococcus Lactis | nisin*² | 430 | 3 × 10\hat{ }7 | 920 | 3 × 10\hat{ }7 |
| | | NCIMB702054 | | | | | |
| 4 | not added | Weissella sp. | PRB | 580 | ND | 1,140 | ND |
| | | AJ110263 | | | | | |
| 5 | not added | Pediococcus pentosaceus | PRB | 450 | ND | 1,064 | ND |
| | | JCM5885 | | | | | |
| 6 | not added | Lactobacillus salivarius | PRB | 460 | ND | 1,176 | ND |
| | | JCM1231 | | | | | |

PRB: Protein Resistant Bacteriosin
BS: Bacillus subtilis IAM1381

### Example 6

The broths obtained by cultivating Lactococcus lactis NCIMB702054 (a strain producing NisinZ) and Weissella sp.AJ110263 (FERM BP-10474) in MRS culture media were adjusted to pH 5.5 with sodium hydroxide. Subsequently, the bacteria were removed from the pH adjusted culture liquid by centrifucation to obtain a supernatant. The broths containing lactic acid bacteria and the supernatants were used in the experiment described below.

5 g of ground meat of Black hair Japanese beef were individually put into six aseptic Falcon tubes (Becton Dickinson Co.50-ml volume) and 5 ml of saline were added to the meat(Sample No.1), 5 ml of 18% salt solution to Sample No.2, 5 ml of the supernatant of Lactococcus lactis NCIMB702054 to Sample No.3, 5 ml of the broth of Lactococcus lactis NCIMB702054 to Sample No.4, 5 ml of the supernatant of Weissella sp. AJ110263 to Sample No.5, 5 ml of the broth of Weissella sp. AJ110263 to Sample No.6. Further, Listeria innocua ATCC33090, statically cultivated in TSBYE medium at 37° C. for 24 hours, was added to the samples in the amount of 10⁸ cfu/ml and the samples were aged at ambient temperature. The samples aged for one day and seven days were collected to count the viable cells of Listeria innocua ATCC33090 in the Listeria selection medium (Oxoid Inc.).

As shown in Table 15, in the saline sample the contaminating bacterium Listeia innocua ATCC33090 existed at 10⁶ cfu/ml or more. In the 18% salt solution sample, the contaminating bacterium Listeia innocua ATCC33090 existed at 10⁵ cfu/ml or more. Where the culture broth or the supernatant of nisin producing lactic acid bacteria were added, the contaminating bacterium Listeia innocua ATCC33090 existed at 10⁶ cfu/ml or more since nisin was decomposed with the protease derived from meat (cathepsin). When using the culture broth or the supernatant of Weissella sp. AJ110263 (FERM BP-10474), however, the contaminating bacterium Listeia innocua ATCC33090 could be reduced to 10³ cfu/ml on the first day of aging up to day 7.

TABLE 15

| | | | | Aging | | Aging | | Aging | |
|---|---|---|---|---|---|---|---|---|---|
| | | | | day 0 | | day 1 | | day 7 | |
| | | Lactic acid | | FAA | | FAA | | FAA | |
| No. | salt | bacterium | bacteriocin | (μmol/g) | cfu/ml | (μmol/g) | cfu/ml | (μmol/g) | cfu/ml |
| 1 | Not added | Not added | | 72 | 2*10\hat{ }6 | 70 | 5*10\hat{ }7 | 100 | 2*10\hat{ }6 |
| 2 | 18% | Not added | | 66 | 4*10\hat{ }6 | 73 | 6*10\hat{ }5 | 70 | 2*10\hat{ }5 |
| 3 | Not added | Lactococcus lactis | nisin Z | 72 | 4*10\hat{ }6 | 75 | 2*10\hat{ }7 | 88 | 4*10\hat{ }6 |
| | | NCIMB702054 sup. | | | | | | | |
| 4 | Not added | Lactococcus lactis | nisin Z | 83 | 4*10\hat{ }6 | 81 | 1*10\hat{ }6 | 84 | 4*10\hat{ }6 |
| | | NCIMB702054 broth | | | | | | | |
| 5 | Not added | Weissella sp | PRB | 82 | 4*10\hat{ }6 | 93 | 4*10\hat{ }3 | 111 | 1*10\hat{ }3 |
| | | AJ110263 sup. | | | | | | | |
| 6 | Not added | Weissella sp | PRB | 84 | 4*10\hat{ }6 | 87 | nd | 104 | 2*10\hat{ }2 |
| | | AJ110263 broth | | | | | | | |

PRB: Protein Resistant Bacteriosin
FFA: Free Amino Acid
Sup: supernatant

Additionally, the antimicrobial spectra were examined. It was indicated that the bacteriocin had a growth-inhibiting effect over Listeria causing food poisoning and Bacillus subtilis being disadvantageous in the manufacturing process of producing soy sauce and miso paste, besides Enterococcus faecium.

Stability against pH and temperature was also examined. The novel bacteriocin retained about 50% of the activity even at pH 2 to 4. In a wide range of pH 2 to pH 11, the antimicrobial activity was stable. Particularly, the bacteriocin had a strong antimicrobial activity around pH 4 to pH 6. Additionally even after heating at 100° C. for 10 minutes, the bacteriocin retained about 50% of the activity. Thus, it was shown that the bacteriocin had great thermal stability.

Numerous modifications and variations on the present invention are possible in light of the above teachings. It is, therefore, to be understood that within the scope of the accompanying claims, the invention may be practiced otherwise than as specifically described herein.

1. A method for producing a lactic acid bacterium culture containing bacteriocin which is resistant to proteases, comprising culturing the lactic acid bacterium for a time and under conditions suitable for expressing said bacteriocin which is resistant to proteases in a suitable medium for said culturing.

2. The method according to claim 1, wherein the lactic acid bacterium belongs to a genus selected from the group consisting of Weissella, Pediococcus, Lactobacillus and Leuconostoc.

3. The method according to claim 2, wherein the lactic acid bacterium belongs to the genus Weissella and is selected from the group consisting of Weissella sp. FERM BP-10474, Weissella cibaria JCM12495, Wissella confusa JCM1093, Weissella hellenica JCM10103, Weissella kandleri JCM5817, Weissella minor JCM1168, Weissella paramesenteroides JCM9890, and Weissella thailandensis JCM10694.

4. The method according to claim 2, wherein the lactic acid bacterium belongs to the genus Pediococcus and is Pediococcus pentosaceus.

5. The method according to claim 2, wherein the lactic acid bacterium belongs to the genus Lactobacillus is selected from the group consisting of Lactobacillus plantarum, Lactobacillus salivarius, and Lactobacillus pentosus.

6. The method according to claim 2, wherein the lactic acid bacterium belongs to the genus Leuconostoc is selected from the group consisting of Leuconostoc citreum, Leuconostoc pseudomesenteroides, Leuconostoc argentinum, Leuconostoccarnosum, and Leuconostoc mesenteroides.

7. A method for preserving a food product, comprising mixing the lactic acid bacterium culture according to claim 1 with a food product during the production thereof.

8. The method according to claim 7, wherein said mixing comprises mixing the lactic acid bacterium culture with the starting materials for production of the food product.

9. The method according to claim 7, wherein said mixing comprises mixing the lactic acid bacterium culture with the final prepared food product.

10. The method according to claim 7, wherein the food product is a fermented food product.

11. The method according to claim 10, wherein said fermented food product is selected from the group consisting of soy sauce, fish sauce, sake, soybean paste miso, pickles, and cheese.

12. The method according to claim 7, wherein the food product is a processed meat product.

13. The method according to claim 1, further comprising recovering the lactic acid bacterium and isolating said bacteriocin which is resistant to proteases.

14. The method according to claim 13, wherein the lactic acid bacterium belongs to a genus selected from the group consisting of Weissella, Pediococcus, Lactobacillus and Leuconostoc.

15. The method according to claim 14, wherein the lactic acid bacterium belongs to the genus Weissella and is selected from the group consisting of Weissella sp. FERM BP-10474, Weissella cibaria JCM12495, Wissella confusa JCM1093, Weissella hellenica JCM10103, Weissella kandleri JCM5817, Weissella minor JCM1168, Weissella paramesenteroides JCM9890, and Weissella thailandensis JCM10694.

16. The method according to claim 14, wherein the lactic acid bacterium belongs to the genus Pediococcus and is Pediococcus pentosaceus.

17. The method according to claim 14, wherein the lactic acid bacterium belongs to the genus Lactobacillus and is selected from the group consisting of Lactobacillus plantarum, Lactobacillus salivarius, and Lactobacillus pentosus.

18. The method according to claim 14, wherein the lactic acid bacterium belongs to the genus Leuconostoc and is selected from the group consisting of Leuconostoc citreum, Leuconostoc pseudomesenteroides, Leuconostoc argentinum, Leuconostoccarnosum, and Leuconostoc mesenteroides.

19. A method for preserving a food product, comprising mixing the said bacteriocin which is resistant to proteases of claim 13 with a food product during the production thereof.

20. The method according to claim 19, wherein said mixing comprises mixing said bacteriocin which is resistant to proteases with the starting materials for production of the food product.

21. The method according to claim 19, wherein said mixing comprises mixing said bacteriocin which is resistant to proteases with the final prepared food product.

22. The method according to claim 19, wherein the food product is a fermented food product.

23. The method according to claim 22, wherein said fermented food product is selected from the group consisting of soy sauce, fish sauce, sake, soybean paste miso, pickles, and cheese.

24. The method according to claim 19, wherein the food product is a processed meat product.

25. A method for screening a lactic acid bacterium which produces bacteriocin which is resistant to proteases comprising
(a) culturing the lactic acid bacterium for a time and under conditions suitable for expressing a bacteriocin in a suitable medium for said culturing to obtain a lactic acid bacterium culture;
(b) assessing the antimicrobial activity of the lactic acid bacterium culture in the presence of a protease; and
(c) classifying the lactic acid bacterium obtained by (a) as resistant to proteases where the culture forms an inhibitory zone in (b).

26. The method according to claim 25, wherein said protease is derived from Aspergillus oryzae.

27. The method according to claim 25, wherein said protease is Umamizyme G.

28. The bacterial strain was defined as Weissella sp. AJ110263 (FERM BP-10474).
